(19) United States
(12) Patent Application Publication
(10) Pub. No.: US 2009/0030516 A1
(43) Pub. Date: Jan. 29, 2009

(54) Surgical Implant With Extracortical Support for a Ligament Transplant

(76) Inventor: Pierre Imbert, Frejus (FR)

Correspondence Address: JAMES C. LYDON, 100 DAINGERFIELD ROAD, SUITE 100, ALEXANDRIA, VA, 22314, US

(21) Appl. No.: 12/223,589

(22) PCT Filed: Nov. 29, 2006
(86) PCT No.: PCT/FR2006/002611

§ 371 Date: Aug. 12, 2008

(30) Foreign Application Priority Data
Nov. 29, 2005 (FR) 0512075

(51) Int. Cl. A61F 2/08

(52) U.S. Cl. 623/13.14

(57) ABSTRACT

A surgical implant (22) used to fix a ligament transplant (18) adapted to connect two bones on either side of a joint. The ligament transplant is fixed in a tunnel (10, 16) pierced from the outer cortical wall of a first bone and extending through a pre-determined depth of a second bone. The implant includes a first end (24) to be fixed to the transplant and a second end in the form of a hook (26) adapted to rest on an outer periphery (28) of the first bone's cortical part at the tunnel's inlet. The hook includes two lugs (30 and 32) adapted to be disposed on either side of the tunnel inlet so the hook rests on solid bone and not on bone having a reduced thickness at the tunnel's position, the axes of the lugs preferably being orthogonal to the hook's plane. One such implant is particularly suitable for replacing the knee's anterior cruciate ligament.

### TECHNICAL FIELD

This invention concerns the field of surgical interventions destined to replace a ligament in a joint and concerns in particular the surgical implant with extracortical support for a ligament transplant.

### STATE OF THE ART

The joints between two bones generally comprise one or more ligaments connecting the two bones on both sides of the joint. The significant force to which these ligaments are subjected can unfortunately cause their rupture. This is the case of the crossed knee ligaments which are highly worked during intense sports like football.

When a ligament such as the anterior cruciate ligament of the knee ruptures, it is possible to replace it by performing a surgical operation called ligamentplasty which consists of fixing a new ligament called “transplant” at the ends of a tunnel extending over both sides of the joint. The tunnel has a length of around 10 cm and has a diameter ranging between 7 mm and 12 mm is pierced into one of the two bones and extends to the other bone at a sufficient depth. The replacement ligament may be an artificial ligament or a portion of a ligament withdrawn from another part of the body or from a cadaver.

Fixation of the transplant through the ends of the tunnel must be solid due to the fact that it constitutes the weak point in the ligament reconstruction in the first post operative weeks. Then, the bone regrowth around the transplant will retain it in a solid and definitive manner (osteointegration phase).

The strength of the initial fixation thus depends on the fixation means. It must be able to resist rupture in traction as in the slippage of the ligament graft with respect to the fixation. Moreover, the attachment system must make it possible to reduce to the maximum the length of the transplant in order to avoid an elastic distension of the transplant which would occur if its length were very significant.

One of the fixation means used usually consists in placing an interference screw at each end of the tunnel. The screw introduced into the tunnel at the same time as the transplant is conical in shape and compresses the transplant inside the tunnel. Unfortunately, the screws rest upon the spongy bone which has less resistance. Moreover, the interference screw may, because of its aggressive threads, damage the transplant at the time it is grafted.

Another means described in document EP 114 6834B consists of using a hook at the lower end of the transplant retained by the cortical part of the bone. But this simple hook is not sufficient insofar as it is retained by the narrowest part of the bone at the inlet of the tunnel.

### PRESENTATION OF THE INVENTION

This is why the purpose of the invention is to use as a fixation means for a transplant, a transplant fixed implant which rests on the solid parts of the external rim of the bone found at the inlet of the tunnel.

The object of the invention is thus a surgical implant which is used to fix a ligament transplant that is intended to replace a ligament connecting two bones on either side of a joint. The ligament transplant is fixed in a tunnel which is pierced from the outer cortical wall of the first bone and which extends through a pre-determined depth of the second bone.

The implant includes a first end which is fixed to the transplant and a second end in the form of a hook which rests on the outer periphery of the cortical part of the first bone at the inlet to the tunnel. The aforementioned hook comprises two lugs which are intended to be disposed on either side of the tunnel inlet such that the hook rests on solid parts of the bone and not on the part having a reduced thickness at the position of the tunnel, the axes of said lugs preferably being orthogonal to the plane of the hook.

### BRIEF DESCRIPTION OF THE FIGURES

The purposes, objects, and characteristics of the invention will appear more clearly upon reading the description made hereafter in reference to the drawings among which:

FIG. 1 shows a cross cut view of the lower end of the femur and the upper end of the tibia showing the tunnel in which the ligament transplant is fixed using an implant resting on the extracortical area;

FIG. 2 shows a view of the face of the lower end of the femur and the upper end of the tibia in place showing a partial cross cut view of the transplant illustrated in FIG. 1.

FIGS. 3A, 3B, and 3C show the stages of implementing a ligament transplant and its fixation using an implant that rests on the endocortical area;

FIG. 4 shows a cross cut view of the lower end of the femur and the upper end of the tibia showing the tunnel in which the ligament transplant is fixed using an implant resting on the extracortical area;

FIG. 5 shows a cross cut view of the lower end of the femur and the upper end of the tibia showing the tunnel in which the ligament transplant is fixed using two non-parallel pins that pass through the transplant;

FIG. 6 shows the ancillary device used to pierce the tunnels designed to receive the two nonparallel pins;

FIG. 7 is a cross-cut view on two planes of the ancillary device showing the two drills used to pierce the tunnels designed to receive the two pins; and

FIG. 8 is a cross-cut view on two planes of the bone and the first branch of the ancillary device showing the implants in the form of pins placed in the tunnels pierced using drills.

### DETAILED DESCRIPTION OF THE INVENTION

The description which follows relates to a surgical intervention having dealt with a ligamentplasty designed to replace one of the anterior cruciate ligaments of the knee connecting the femur and the tibia. But it goes without saying that this intervention could also be used to replace a ligament found in another joint that the knee.

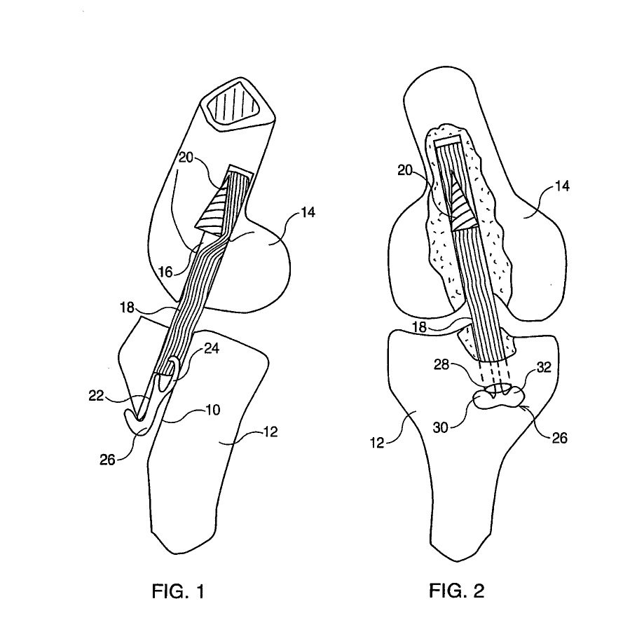

As illustrated in FIGS. 1 and 2, a tunnel (10) with a diameter ranging between 7 mm and 12 mm, were pierced starting from the cortical wall of the tibia (12) and crossing to the upper end of the tibia. The tunnel (10) extends into the femur (14) through the portion of the tunnel (16) over a pre-determined length.

A transplant (18) which can be an artificial ligament or a portion of a ligament taken from another part of the body of the patient or from a cadaver is introduced into the tunnel (10) and its extension (16). An interference screw (20) was introduced after the transplant into the prolongation of the tunnel (16) in order to block the upper end of the transplant. As can be seen in FIG. 1, the interference screw (20) compresses the ligament transplant against the wall of the tunnel and overflows from the tunnel due to the fact that the part of the bone in which the tunnel was pierced is the spongy part of the bone. It should be noted that any other means of fixation may be used in place of the interference screw and in particular the means of fixation described below.

At the lower end of the tunnel, the ligament transplant (18) comprises a solid rigid implant (22) of which the upper part (24) is in the form of a loop through which it is made to pass the transplant which is generally folded upon itself before introducing it into the tunnel. The lower portion (26) of the implant (22) is bent to form a kind of hook. When the transplant (18) is introduced and pushed into the tunnel, the hook (26) rests against the external rim (28) of the inlet to the tunnel (10). This external rim is made up of the cortical wall of the bone; it is rigid and holds the implant (22) when a traction force is exerted on the ligament transplant being pulled upwards before being fixed definitively by the interference screw (20).

However, a simple hook will be held by the part of the bone that has a reduced-thickness at the inlet of the underlying tunnel which runs the risk of breaking under the effect of the force exerted in traction. To avoid this disadvantage, the hook (26) as shown in FIG. 2, includes two lugs (30 and 32) which are located on both sides of the tunnel inlet in such a way that the hook rests on solid parts of the bone, the axes of said lugs preferably being orthogonal to the plane of the hook.

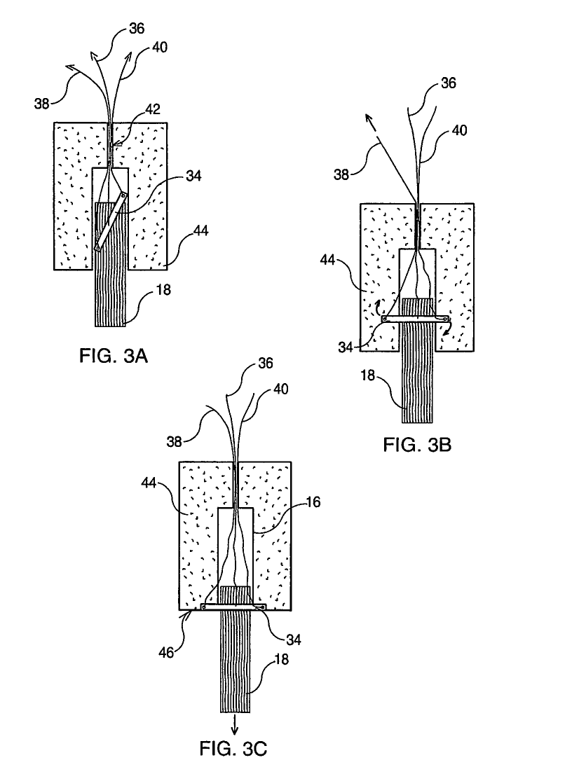

As mentioned previously, the means of fixation blocks the upper part of the transplant and may be carried out differently from the illustration in FIGS. 3A, 3B, and 3C. In that case, a stem (34) is fixed at its midpoint to the upper part of the ligament transplant (18). This stem has a length that is greater than the diameter of the tunnel, for example 16 mm if the tunnel's diameter is 10 mm. Three flexible wire are fixed at the stem (34). The first wire (36) is fixed at the midpoint of the stem (which is itself fixed at the transplant) and two other wires (38 and 40) are fixed at the two ends of the stem. When the tunnel (16) was pierced into the femur, a mini tunnel (42) was also pierced to the cortical wall of the bone. The three wires (36, 38, and 40) are introduced through the mini tunnel (42) until they exit at the end of the latter. It is then easy to pull the wire (36) to bring the ligament transplant to the end of the tunnel (16) all while holding the stem in the axis of the tunnel because of the wire (40), in such a way that it remains entirely between the walls of the tunnel.

When the transplant (18) was fully introduced into the tunnel, traction is exerted on the wire (38). As shown in the FIG. 3B, this traction tends to make the stem (34) swivel around the point of its fixation to the transplant until it occupies a position perpendicular to the axis of the tunnel. This is relatively easy since both ends of the stem easily penetrate the spongy part of the bone (44) shown schematically in the figures.

Then the force of traction to the bottom is exerted on the transplant (18) as illustrated in FIG. 3C so that stem (34), as it remains in its transverse position, goes down to the bottom of tunnel (16) while being inserted into the spongy part of the bone until it reaches the interior surface of the rigid and hard cortical wall (46) in the base of the femur (14).

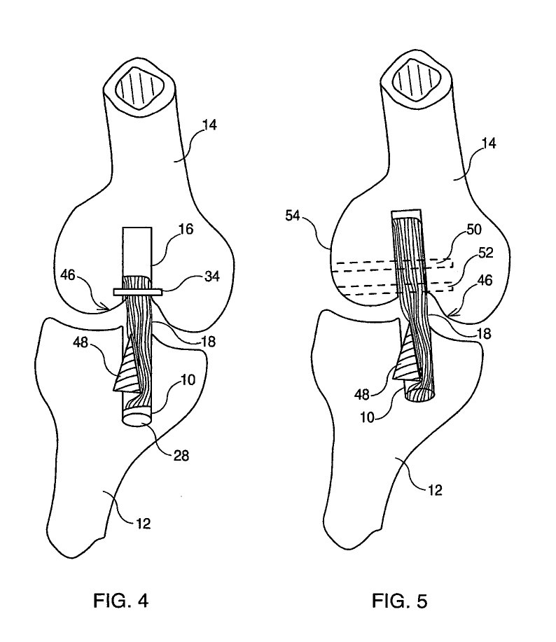

As can be seen in FIG. 4, the stem (34) remains as a support on the cortical wall (46) of the femur and in this way preserves the firm fixation of the transplant (18). After this operation, the lower part of the transplant (18) is fixed in the tunnel using an interference screw (48) introduced through the inlet of the tunnel (28). It should be mentioned that fixation means other than the interference screw may be used.

Another fixation technique for the upper part of the transplant in its tunnel is now described in reference to FIGS. 5, 6, 7, and 8. As illustrated in FIG. 5, the transplant is fixed in its upper part using implants having the form of pins (50 and 52) that cross through the transplant (18) and that are introduced into the cortical part (54) of the femur (14) as can be seen below. The peculiarity of these pins is that they are not parallel but between them form an acute angle and that the lowest pin is supported on the interior surface of the rigid cortical wall (46) at the base of the femur (14). As before, the lower part of the transplant is fixed by an interference screw (48) or even another means of fixation such as the implant illustrated in FIG. 1.

It should be noted that this fixation technique using pins may be used to fix the lower part of the transplant, for example when the upper part of the transplant is fixed by a transverse stem as illustrated in FIG. 4.

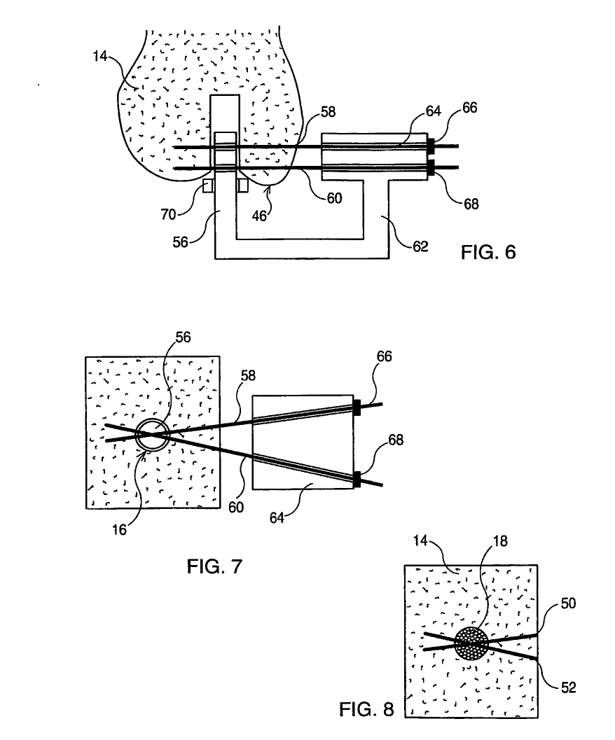

Piercing the tunnels destined to receive the pins (50 and 52) is made using an ancillary device illustrated in FIG. 6. This device is U-shaped and includes a first branch (56) introduced into the portion of the tunnel (16) located in the femur (14). This branch is pierced with two transverse tunnels located at two different heights and destined to be crossed by two drill bits (58 and 60).

At the top of the second branch of the device (62) is a pierced plate (64) with two transverse tunnels situated at different heights and forming between them an acute angle as seen in FIG. 7. The two drills (58 and 60) introduced into the two tunnels in the plate (64) are used to pierce two tunnels in the femur for the purpose of introducing the two implants in the form of pins (50 and 52).

When the two drills (58 and 60) finish piercing, they have crossed the branch (56) and are blocked by two supports (66 and 68) against the plate (64).

The branch (56) is threaded and includes an equally threaded nut (70) that may be go down or up along the branch (56) in such a way that the drill (60) pierces a tunnel in the femur just to the top of the internal surface of the rigid cortical wall (46) of the bone when the nut is supported against the external surface of the rigid cortical wall (46).

The two drills are then withdrawn and the implants in the form of pins (50 and 52) are introduced in the tunnels that have been pierced as illustrated in FIG. 8. The two pins thus form an acute angle between them and the pin (52) located right at the top of the cortical wall is thus blocked. Supposing that the tunnel is higher than the cortical wall, the pins that are mainly found in the spongy part of the bone will need to be lowered under the effect of the traction exerted by the transplant until the pin (52) reaches the cortical wall, but this is difficult in insofar as, (since the pins are not parallel), they are located in different vertical planes.

1-3. (canceled)

4. Surgical implant adapted to fix a ligament transplant connecting two bones on either side of a joint, wherein said ligament transplant is fixed in a tunnel formed by a first portion pierced from the exterior cortical wall of a first bone and extending through a second portion at a pre-determined depth of a second bone, said implant including a first end adapted to be fixed by said transplant and a second end in the form of a hook destined to be supported against an outside rim of the cortical part of the said first bone found at the inlet of said first portion of the tunnel;
wherein said hook comprises two lugs which are adapted to be located on either side of said tunnel inlet such that the hook rests on solid parts of the bone and not on bone having a reduced thickness at the position of the tunnel.

5. The surgical implant of claim 4, wherein the axes of said lugs are orthogonal to a plane of the hook.

6. The surgical implant of claim 4, wherein said first end is in the form of a loop through which said transplant is passed folded up on itself before introducing it into said tunnel.

7. Surgical fixation set for a ligament transplant fixed at one end by an implant according to claim 4, further comprising a means of fixation from the other end of said transplant constituted by two pins adapted to be fixed transversally on said transplant, the two pins being non-parallel and located on two different planes, the lower pin is supported against the interior surface of the cortical part of the second bone.

8. Surgical fixation set for a ligament transplant fixed at one end by an implant according to claim 6, further comprising a means of fixation from the other end of said transplant constituted by two pins adapted to be fixed transversally on said transplant, the two pins being non-parallel and located on two different planes, the lower pin is supported against the interior surface of the cortical part of the second bone.
